(19) United States
(12) Patent Application Publication
(10) Pub. No.: US 2002/0061514 A1
(43) Pub. Date: May 23, 2002

(54) Assay for identifying modulators of chondrogenesis

(76) Inventors: Tully Michael Underhill, London (CA); Andrea Dawn Weston, St. Mary's (CA)

Correspondence Address: Micheline Gravelle, Bereskin & Parr, Box 401, 40 King Street West, Toronto, ON, M5H 3Y2, CA

(21) Appl. No.: 09/957,456

(22) Filed: Sep. 21, 2001

Related U.S. Application Data
(60) Provisional application No. 60/234,242, filed on Sep. 21, 2000.

(51) Int. Cl. C12Q 1/00; C12Q 1/68

(52) U.S. Cl. 435/4; 435/6

(57) ABSTRACT

A screening assay for identifying modulators of chondrogenesis is described. The assay comprises providing cells capable of differentiating into chondroblasts; transfecting the cells with a nucleic acid construct comprising a reporter gene that is detectable upon chondroblast differentiation; adding a test compound to the transfected cells; and determining the effect of the test compound on chondroblast differentiation. The reporter gene is preferably responsive to the transcription factor Sox9. The modulators may be useful as therapeutic agents that can inhibit or enhance cartilage formation. The assay may also be used to diagnose, monitor or prognose diseases associated with abnormal cartilage formation.

This application claims the benefit under 35 USC §119(e) from U.S. Provisional patent application serial No. 60/234,242, filed Sep. 21, 2000.

### FIELD OF THE INVENTION

The present invention relates to assays, nucleic acid constructs and kits for identifying modulators of chondrogenesis.

### BACKGROUND OF THE INVENTION

Identifying the intrinsic and extrinsic factors that regulate the commitment and differentiation of multipotential cells is critical to gaining a better understanding of developmental processes. Using various molecular biological approaches it is possible to identify putative regulators of commitment and differentiation. For instance, the recent development of DNA microarray technology has made it possible to rapidly identify hundreds of genes whose expression changes during development. This technology is very powerful and will greatly facilitate attempts to understand developmental events at a molecular level. However this, and other technologies that examine differential gene expression, specify the need for an easy and rapid functional assay to assess the process-specific relevance of several genes at once. Given that cell differentiation is the end-point of many developmental processes it would be beneficial to have a method available to quickly assess the ability of various genes to modulate cell differentiation. Not only would such a technology allow researchers to identify pathways involved in cell differentiation, but it would also serve as a useful tool for dissecting those pathways and for deciphering the hierarchies of the molecules involved.

Within the developing limb many of the signals involved in regulating pattern formation have been elucidated. For example, sonic hedgehog (SHH), bone morphogenetic proteins (BMPs), fibroblast growth factors (FGFs) and WNTs are all signals that figure prominently in determining or specifying pattern (12). The mechanism whereby these signals converge to regulate the aggregation of chondroprogenitors into precartilaginous condensations, however, is poorly defined. Following condensation, cells within the central part of the condensate differentiate into chondroblasts (11). This is accompanied by the upregulation of various genes including type II collagen (col II) whose expression increases dramatically upon differentiation.

Chondroblast differentiation and cartilage formation are reliant upon the transcription factor Sox9. Animals deficient in Sox9 are embryonic lethal and Sox9-null cells do not contribute to cartilage (3). Sox9 binding sites have been located within the first intron of col II and have been found to be important in regulating its expression (13, 20, 22). Sox9 is weakly expressed in condensed mesenchymal cells and is quickly upregulated during chondroblast differentiation. In this context, Sox9 activity reflects the differentiation status of chondroblasts and, indirectly, the commitment of skeletal progenitors to the chondrocytic lineage. The activity of this transcription factor therefore, provides an accurate measure for efficiently assessing skeletal progenitor commitment and differentiation (15).

Osteoarthritis is a condition that results from the breakdown of articular cartilage. Approximately 20.7 million Americans are affected by this painful disease and almost everyone suffers from it by age 60. Cartilage has very little ability to repair itself. Consequently, there is a tremendous need for the identification of genes and/or compounds that stimulate cartilage formation.

Thousands of genes have been identified but functional data is available for only a small subset of these genes. There is a continuing need for efficient technologies that can be used to aid in the development of drug targets or drug candidates by identifying the function of genes and/or compounds.

### SUMMARY OF THE INVENTION

The present inventors have developed a rapid and efficient screening assay for identifying modulators of chondrogenesis or skeletogenesis. The assays are useful in the identification of therapeutic agents that can modulate cartilage formation, in developing diagnostic or prognostic assays for assessing the status of diseases associated with a modulation of chondrogenesis or for gene discovery purposes in understanding skeletal development and cartilage formation.

Accordingly, the present invention provides a method of identifying a modulator of chondrogenesis comprising:

(a) providing cells capable of differentiating into chondroblasts or chondrocytes;

(b) transfecting the cells with a nucleic acid construct comprising a reporter gene that is detectable upon chondroblast or chondrocyte differentiation;

(c) adding a test compound to the transfected cells; and

(d) determining the effect of the test compound on chondroblast or chondrocyte differentiation.

In one embodiment the test compound is a nucleic acid construct comprising a test gene that is co-transfected into cells that are capable of differentiating into chondroblasts or chondrocytes.

The present invention also includes a kit for use in identifying a modulator of chondrogenesis comprising a nucleic acid construct comprising a reporter gene that is capable of detecting chondroblast or chondrocyte differentiation and instructions of use thereof. Preferably, the reporter is the pGL3(4X48)-luciferase or pGL3(4X48)-EGFP as shown in FIG. 6.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is an overview of the reporter gene cell differentiation assay of the invention. The method used to transfect limb mesenchymal cells, and the assay for reporter activity are illustrated in (A). Bright-field images of day 4 cultures are shown at low magnification (B) and high magnification (E), note the abundance of cartilage nodules. Corresponding images taken under epifluorescence demonstrate an abundance of EGFP-expressing cells throughout the culture (C, F). Note the distribution of EGFP-expressing cells both within cartilage nodules, and in the periphery. EGFP-expressing cells at the periphery of the cultures have a morphology that is fibroblastic-like (D), whereas cells within the nodules appear oval, consistent with the expected chondroblast phenotype (G). Scale bar: 1200 μm (B, C); 300 μm (E, F) and 600 μm (D, G).

FIG. 2 is a bar graph showing the comparison of basal reporter activities in primary limb mesenchymal cells. Basal activities of pW1-col II-Luc and 4X48-p89 are low compared to the activity of the pGL3(4X48) reporter. The higher levels of basal activity provided by the pGL3(4X48) reporter makes it the preferred reporter for analyzing factors that inhibit reporter activity. (ANOVA, P<0.0001; Bonferonni post-tests: P<0.001).

FIG. 3 shows the chondrogenic stimulatory activities of BMP-2 and -4. Untreated control cultures were stained with alcian blue following 2 and 4 days of incubation, respectively (A, B). Cultures were treated with 10 ng/ml BMP-2 for 2 and 4 days (C and D respectively), or BMP-4 (E, F), fixed, then stained with alcian blue. In response to BMP-2 and -4 there is an increase in both nodule number and average nodule size with the effects of BMP-4 being most dramatic. Addition of BMP-2 stimulates precocious expression of Sox9 and col II is indicated by Northern blot analysis (G). Both BMP-2 and BMP-4 stimulate activity of the pGL3(4X48) reporter, albeit the effects of BMP-4 are more pronounced (H). Co-transfection of constitutively active BMP receptors, Alk3 (Alk3*) and Alk6 (Alk6*) also induces reporter gene activity (H). Bar: 1 mm (A-F). (ANOVA P<0.001; Bonferroni tests: *P<0.05, **P<0.001).

FIG. 4 shows the analysis of Sox9 requirement for activation of pGL3-4X48-EGFP. Cos P7 cells were transfected with pGL3-4X48-EGFP and either empty vector (A, C), or a Sox9 expression vector (B, D). A and B correspond to bright field images, whereas C and D are epifluorescent images. Only those cells transfected with a pcDNA-Sox9 activate the reporter gene (D). Primary limb mesenchymal cultures were transfected with pGL3-4X48-EGFP and examined 64 hours post-transfection (E, F). In response to 10 ng/ml BMP-4 (F), there is an increased number of EGFP-expressing cells compared to untreated control cultures (E), indicating that BMP-4 induces chondrogenesis by enhancing the recruitment of cells to the chondrogenic lineage. An image representative of that observed in several independent cultures is shown. Scale bars: 300 μm (A-D) and 600 μm (E, F).

FIG. 5 shows the differential use of developing limb regions to enrich myogenic or chondrogenic precursor populations. In cells isolated from the distal tip of the limb bud there is considerably higher basal activity of the pGL3(4X48) reporter (A), reflecting a greater proportion of chondroprogenitors in this region. In contrast, muscle-specific reporters such as pGL3-myogenin-Luc, pGL2-E4-Luc, and pGL3-c-actin-Luc have a much higher activity in cells from the proximal region of the limb bud due to the presence of more myogenic precursors. The differences in chondrogenic and myogenic populations between proxial and distal cultures is revealed by the presence of greater alcian blue-positive cartilage nodules in the distal cultures (C) compared to the proximal cultures (D), and by a greater number of MyHc-positive myocytes in proximal cultures (F) compared to distal cultures (E). Bars: (C, D) 2.0 mm; (E, F) 1.2 mm. (Student's t-tests: *P<0.01. **P<0.05).

FIG. 6 is a schematic representation of the pGL3(4X48) reporter gene construct.

### DETAILED DESCRIPTION OF THE INVENTION

I. Screening Assay

The present inventors have developed a rapid and efficient screening assay for identifying modulators of chondrogenesis. The assays are useful in the identification of therapeutic agents that can modulate cartilage formation, in developing diagnostic or prognostic assays for assessing the status of diseases associated with a modulation of chondrogenesis or for gene discovery purposes in understanding skeletal development and cartilage formation.

Accordingly, the present invention provides a method of identifying a modulator of chondrogenesis comprising:

(a) providing cells capable of differentiating into chondroblasts or chondrocytes;

(b) transfecting the cells with a nucleic acid construct comprising a reporter gene that is detectable upon chondroblast or chondrocyte differentiation;

(c) adding a test compound to the transfected cells; and

(d) determining the effect of the test compound on chondroblast or chondrocyte differentiation.

The cells capable of differentiating into chondroblasts or chondrocytes can be any cells that can, in the appropriate conditions, differentiate into chondroblasts or chondrocytes. In a preferred embodiment, the cells are limb mesenchymal cells, or cell lines established from mesenchymal cells, mesenchymal stem cells, or cell lines established from chondrocytes. The cells can be obtained from any animal species including mice, rats, rabbits and humans. In one embodiment, limb mesenchymal cells are obtained from the early stage embryonic limb buds (E10-E 12) of mice.

In a preferred embodiment, the cells are plated at high density. When plated at high density, limb mesenchymal cells at the appropriate embryonic stage form cartilage nodules in vitro in a manner that closely follows the in vivo process (1). Moreover, these cells are robust, easy to isolate, and are pluripotent. Shortly after seeding, the cells form a confluent monolayer with precartilaginous condensations evident within 24 hours. After approximately 48 hrs, small cartilage nodules appear that weakly stain with alcian blue, and cells weakly express Sox9 and col II. Following an additional 24 - 48 hrs there is a dramatic upregulation of Sox9 and col II expression which is closely associated with an increase in the number and size of cartilage nodules that stain intensely with alcian blue.

The reporter gene can be any gene that can be used to detect or measure chondroblast differentiation or chondrogenesis. In one embodiment, the reporter gene contains a sequence that binds to a protein that is upregulated during chondrogenesis. In particular, the reporter gene may comprise one or more enhancer elements that bind to a transcription factor that is upregulated during chondroblast differentiation. Examples of transcription factors that are upregulated during chondroblast differentiation include Sox9, Sox5 and Sox6. The enhancer element region may comprise a single or reiterated transcription factor binding site or may comprise a mixture of binding sites for various transcription factors.

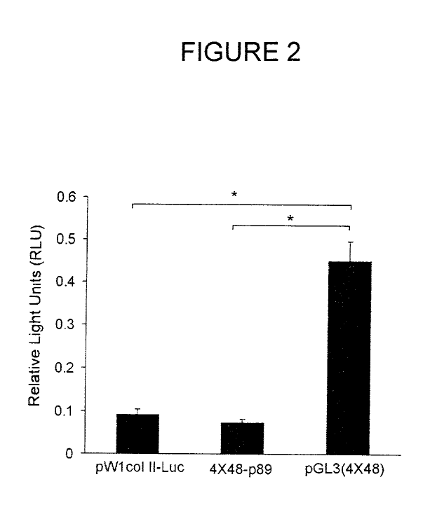

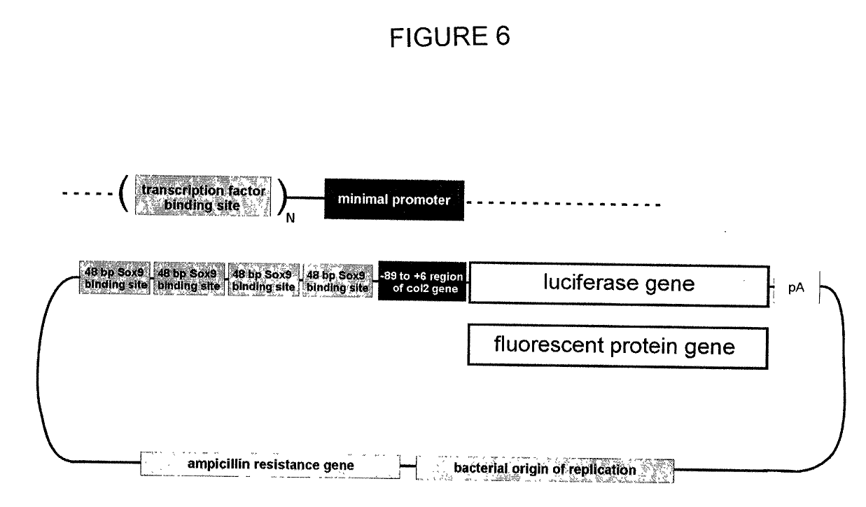

In a particular embodiment, the reporter gene is responsive to the transcription factor Sox9. In this regard, the promoter gene will contain at least one Sox9 binding site. In a specific embodiment, the reporter gene contains four 48 bp Sox9 binding sites. More preferably, the construct is pGL3(4X48)-luciferase or pGL3(4X48)-EGFP shown in FIG. 6. The inventors have shown that the pGL3(4X48) reporter responds dramatically to an increase in Sox9 (FIG. 2A), and has the added benefit of having a much higher output in untreated or unmodified cells (2B) enabling the study of genes or factors that decrease Sox9 activity.

The nucleic acid construct comprising the reporter will also comprise a promoter sequence such as the col 11 promoter region or derivatives thereof. Other promoters that may be used include the aggrecan promoter or derivatives thereof or other promoters for genes that are expressed in chondrocytes.

The nucleic acid construct comprising the reporter gene preferably also comprises a detectable marker such as a gene encoding luciferase or enhanced green fluorescent protein. Preferably the luciferase gene is a modified form that makes it more stable and better suited for screening assays. Examples of modifications include: modifying sequences in the luciferase gene to eliminate the peroxisomal targeting sequence; introducing Kozak consensus sequence immediately 5′ of the translational initiation site of the luciferase gene to improve translation; and replacing the early SV40 polyadenylation sequence at the 3′ end of the luciferase gene with the late SV40 polyadenylation sequence. Preferably the luciferase gene is the modified form provided in pGL3-Basic (Promega). Other markers that may be used include green fluorescent proteins or derivatives thereof (such as enhanced green fluorescent protein), red fluorescent proteins, other fluorescent proteins, other types of luciferases and genes whose product or activity is easily assayable. The nucleic acid construct may also contain an antibiotic resistance gene such as an ampicillin resistance gene and a bacterial origin of replication.

The phrase “determining the effect of the test compound on chondroblast or chondrocyte differentiation” means that the effect of the test compound on the activity of the reporter gene will be assayed and compared to the activity that is normally observed in the absence of the test compound. Preferably the screening assay is repeated using a control sample with the same conditions and components as the test sample but without the test compound. The activity of the reporter gene in the test sample is then directly compared to the control. The assay for determining the activity of the reporter gene will depend on the nature of the detectable marker in the construct. When the marker is luciferase, the luciferase activity is measured using known assays.

The cells are preferably transfected in a transient transfection system. Transient transfections allow for the potential analysis of hundreds to thousands of genes and/or compounds within a single experiment. In addition, when using transient transfections instead of retroviral or adenoviral vectors the time-consuming need to generate and package the virus is eliminated, again making the assay amenable to high-throughput screening. In addition, the inventors have developed optimal conditions under which we obtain a sufficient number of transfected cells within a relatively short period of time, allowing one to monitor cell fate changes that occur very early in the culture period.

Preferably the nucleic acid construct containing the chondrogenic reporter gene and a nucleic acid containing a reporter gene allowing for normalization of transfection efficiency (ie. pRLSV40) are mixed and the transfection reagent added. This mixture is added to the cells prior to plating as such the approach greatly enhances the efficiency of the transfection and leads to a random distribution of transfected cells in the culture.

The test compound can be any compound which one wishes to test including, but not limited to, proteins, peptides, nucleic acids (including RNA, DNA, antisense oligonucleotide, peptide nucleic acids), carbohydrates, organic compounds, natural products, library extracts, bodily fluids and other samples that one wishes to test for modulators of chondrogenesis. When the test compound is a nucleic acid it will be transfected into the cells before, during or after transfection with the nucleic acid construct containing the reporter gene. In one embodiment, the test gene may be present in the same nucleic acid construct as the reporter gene. In another embodiment, the test gene is present in a separate nucleic acid construct and contains the necessary regulatory elements for expression of the test compound in the cells.

More than one test compound can be tested at a time in the assay of the invention. As such the assay is useful in testing the combined effects of two or more compounds on chondrogenesis. In particular, the inventors have found that the expression of either cMEKK1 or dnFos results in a decrease in reporter gene activity but when expressed together the reporter gene activity is increased.

The method is adaptable to high-throughput screening applications. For example, a high-throughput screening assay may be used which comprises any of the methods according to the invention wherein aliquots of transfected cells are exposed to a plurality of test compounds within different wells of a multi-well plate. Further, a high-throughput screening assay according to the invention involves aliquots of transfected cells which are exposed to a plurality of candidate factors in a miniaturized assay system of any kind. Another embodiment of a high-throughput screening assay could involve exposing a transfected cell population simultaneously to a plurality of test compounds. Also, transfecting a cell population with a plurality of different constructs having different reporter genes would allow for simultaneous determination of different responses or differentiation patterns.

The method of the invention may be “miniaturized” in an assay system through any acceptable method of miniaturization, including but not limited to multi-well plates, such as 24, 48, 96 or 384-wells per plate, micro-chips or slides. The assay may be reduced in size to be conducted on a micro-chip support, advantageously involving smaller amounts of reagent and other materials. Any miniaturization of the process which is conducive to high-throughput screening is within the scope of the invention.

The simplicity of this assay enables rapid identification of test compounds such as genes, small molecules, drug candidates, etc., that regulate cellular differentiation, such as the chondrogenic program. The invention can be used to efficiently delineate effectors operating downstream or upstream of a gene or factor of interest, allowing one to elucidate the genetic and signalling networks operating to regulate developmental processes. The invention may be used to identify genes that either inhibit or augment chondrogenesis and can be applied to defining mechanisms through which chondrogenic stimulatory factors exert their effects. The invention allows not only identification of those genes involved in a differentiation program, but also allows further understanding of the regulatory network involved in differentiation. The inventive method allows efficient transfection of primary cultures and allows the activity of transfected reporter genes to be observed.

The inventive method may be modified to follow many differentiation processes at once through the use of multiple reporter systems. In this manner, switches controlling decisions between more than one possible cell fate can be identified. The identification of such switches will enhance the understanding of early developmental processes and will greatly facilitate attempts to recapitulate in vivo processes through tissue engineering.

The inventive method may be used to screen several test compounds in a very short period of time. Through the use of various inhibitors and activators of signaling pathways, the invention will allow for the elucidation of the mechanisms through which signaling is achieved. High-throughput screen embodiment of the invention advantageously allows rapid evaluation of factors affecting cellular processes.

As shown in Table 1, the inventors have tested many genes in the assay of the invention and have shown that the method is very effective in identifying modulators of chondrogenesis. While no other groups have described a screening assay for identifying modulators of chondrogenesis, Murakami et al. (15) did test the effect of bone morphogenetic protein-2 (BMP-2) on the differentiation of primary chondrocytes. In contrast to the results shown by the present inventors, Murakami et al. did not see any response upon treatment with BMP-2. In addition, Murakami et al. observe that expression of a constitutively active version of Mek1 (cMek1) promotes reporter gene activity. In contrast, the inventors find that expression of cMek1 (see Table 1) inhibits reporter gene activity. The different results reported by Murakami et al. are likely due to the fact that their assay system is not as sensitive or effective as the assay of the invention.

It is also worth noting that the other groups including Murakami et al. have used a reporter to examine Sox9 expression in chondrocytes. However, they used the reporter to look at factors that upregulate Sox9 in cells that already express Sox9. In the present assay, the inventors are identifying factors that initiate Sox9 expression in cells that do not necessarily express Sox9. Thus, the inventors are using the reporter for a different purpose, and the fact that others obtain different results than the inventors suggests that they are looking at the ability of Sox9 to be modulated at different stages, primarily within chondrocytes. The present assay is directed in part towards identifying factors that initiate Sox9 expression in prechondrogenic cells or mesenchymal cells.

II. Uses of the Assay

The present invention includes all possible uses of the screening assay of the invention, some of which are summarized below.

(a) Modulators of Chondrogenesis

The invention extends to any compounds or modulators of chondrogenesis identified using the screening method of the invention. The term “modulator of chondrogenesis” as used herein means a test compound or substance that can modulate the differentiation of cells into chondrocytes or chondroblasts. The term includes both test compounds or substances that can activate or enhance chondrogenesis as well as test compounds or substances that can inhibit or suppress chondrogenesis. Such modulators include, but are not limited to, proteins, peptides, nucleic acids (including RNA, DNA, genes, antisense oligonucleotides, peptide nucleic acids), carbohydrates, organic compounds and natural products. Some of the modulators of chondrogenesis identified using the method of the invention are shown in Table 1.

The invention also includes a pharmaceutical composition comprising a modulator of chondrogenesis identified using the screening method of the invention in admixture with a suitable diluent or carrier. The invention further includes a method of preparing a pharmaceutical composition for use in modulating chondrogenesis comprising mixing a modulator of chondrogenesis identified according to the screening assay of the invention with a suitable diluent or carrier.

(b) Kits

The development of the screening assay of the invention allows the preparation of kits for use in identifying modulators of chondrogenesis. The kits would comprise the reagents suitable for carrying out the methods of the invention, packaged into suitable containers and providing the necessary instructions for use. For example, the kit may comprise the pGL3(4X48)-luciferase reporter or the pGL3(4X48)-EGFP reporter, pRLSV40 (for normalization of transfection efficiency), transfection reagents and reagents for measuring luciferase activity. The kit may provide instructions for isolating limb mesenchymal cells as well as instructions for carrying out the assay of the invention.

The term “instructions” or “instructions for use” typically includes a description describing the reagent concentration or at least one assay method parameter such as the relative amount of the reagent-sample admixtures, temperature, conditions and the like.

Accordingly, the present invention provides a kit for use in identifying a modulator of chondrogenesis comprising a nucleic acid construct comprising a reporter gene that is capable of detecting chondroblast differentiation and instructions for use thereof. Preferably, the reporter is the pGL3(4X48)-luciferase or pGL3(4X48)-EGFP reporter.

(c) Therapeutic Uses

The assay and kit of the invention allow the identification of modulators of chondrogenesis. Compounds or genes that stimulate chondrogenesis or cartilage formation may be used in developing drugs for treating or preventing diseases and conditions involving cartilage deficiencies. Such diseases and conditions include, but are not limited to, osteoarthritis, chondrodysplasias, chondrolysis, chondromalacia, chondrodystrophies, chondroma, chondromatosis, chondromyxoma, cartilaginous tumors, general chondropathies or other disorders involving abnormal cartilage formation or abnormal skeletal development resulting from disease or trauma. Accordingly, the present invention also provides a method of stimulating cartilage formation comprising administering an effective amount of a compound, nucleic acid containing a vehicle for expression of a gene or a gene-product according to the method of the invention to an animal in need thereof.

The term “effective amount” as used herein is defined as an amount effective, at dosages and for periods of time necessary to achieve the desired result. The effective amount of a compound of the invention may vary according to factors such as the disease state, age, sex, and weight of the animal. Dosage regima may be adjusted to provide the optimum therapeutic response. For example, several divided doses may be administered daily or the dose may be proportionally reduced as indicated by the exigencies of the therapeutic situation.

The term “animal” as used herein includes all members of the animal kingdom, including humans. Preferably, the animal to be treated is a human.

Compounds or genes that inhibit cartilage formation may be useful in treating or preventing diseases wherein a reduction in cartilage formation is desirable such as disorders associated with increased cartilage formation including, but not limited to, fibrodysplasia ossificans progressive, chondrosarcomas, exostoses, and benign and malignant tumors of cartilage.

Accordingly, the present invention provides a method of inhibiting cartilage formation comprising administering an effective amount of a compound, nucleic acid containing a vehicle for expression of a gene or a gene-product identified according to a method of the invention to an animal in need thereof.

The method of the invention may also be used in tissue engineering studies to aid in the identification of genes and their compounds that will stimulate or inhibit cartilage formation. The modulators of chondrogenesis will be useful in tissue generation for the repair of birth defects or for use in reconstructive surgery.

(d) Diagnostics

The screening assay of the invention can be used to diagnose, prognose or monitor certain diseases and/or the effectiveness of the treatment of certain diseases. For example, the assay could be used to detect for the presence of chondrogenic modulators that may be released by cartilage into the synovial fluid or other bodily fluids. Such modulators may result from inflammation of the joint, cartilaginous lesions or wear and tear on joint cartilage. The assay could be used to detect the presence of these factors and as such could be used to assay for cartilage breakdown. During cartilage repair, chondrocytes may release factors into the synovial fluid or blood that would affect chondrocyte differentiation, and the presence of these factors could be detected using the assay.

Therefore the assay of the invention may be used to test bodily fluids for factors (i.e. test compounds) that modulate chondrogenesis. The presence or absence of such factors may be diagnostic or prognostic of certain conditions that are related to a modulation (e.g. increase or decrease) in chondrogenesis or cartilage formation.

Accordingly, the present invention provides a method of diagnosing or monitoring a disease that is associated with a modulation in chondrogenesis comprising:

(a) providing cells capable of differentiating into chondroblasts or chondrocytes;

(b) transfecting the cells with a nucleic acid construct comprising a reporter gene that is detectable upon chondroblast or chondrocyte differentiation;

(c) adding a test sample to the transfected cells; and

(d) determining the effect of the test sample on chondroblast or chondrocyte differentiation.

The disease can be any disease associated with a modulation in chondrogenesis or cartilage formation including, but not limited to, osteoarthritis, chondrodysplasias, chondrolysis, chondromalacia, chondrodystrophies, chondroma, chondromatosis, chondromyxoma, cartilaginous tumors, general chondropathies or other disorders involving abnormal cartilage formation or abnormal skeletal development resulting from disease or trauma, fibrodysplasia ossificans progressiva, chondrosarcomas, exostoses, and benign and malignant tumors of cartilage.

The test sample can be any sample that one wishes to test including bodily fluids such as synovial fluid.

(e) Drug Discovery

The present invention also includes all business applications of the screening assay of the invention including conducting a drug discovery business.

Accordingly, the present invention also provides a method of conducting a drug discovery business comprising:

(a) providing one or more assay systems for identifying a modulator of chondrogenesis;

(b) conducting therapeutic profiling of modulators identified in step (a), or further analogs thereof, for efficacy and toxicity in animals; and

(c) formulating a pharmaceutical preparation including one or more modulators identified in step (b) as having an acceptable therapeutic profile. In certain embodiments, the subject method can also include a step of establishing a distribution system for distributing the pharmaceutical preparation for sale, and may optionally include establishing a sales group for marketing the pharmaceutical preparation.

The present invention also provides a method of conducting a target discovery business comprising:

(a) providing one or more assay systems for identifying modulators of chondrogenesis;

(b) (optionally) conducting therapeutic profiling of modulators identified in step (a) for efficacy and toxicity in animals; and

(c) licensing, to a third party, the rights for further drug development and/or sales for modulators identified in step (a), or analogs thereof.

The following non-limiting examples are illustrative of the present invention:

### EXAMPLES

### EXAMPLE 1

Materials and Methods

Construction of expression and reporter plasmids

To increase reporter gene sensitivity, the Sox9 responsive reporter gene from 4X48-p89 luciferase (14) was subcloned into pGL3-basic (Promega). A fragment containing the reiterated (4 X 48) Sox9 binding sequence upstream of the mouse col II minimal promoter (−89 to +13) was liberated from 4X48-p89 plasmid by digestion with Bam HI and Hind III. This fragment was subcloned into the Bgl II and Hind II sites of pGL3-basic (Promega) to generate pGL3(4X48). To generate an EGFP-based col II reporter, the luciferase gene within pGL3(4X48) was replaced with EGFP-N1 (Clontech). For expression of genes in primary cultures a modified version of pSG5 (Stratagene), pSG5HS, was used which contains two additional restriction endonuclease sites, Hind III and Spe I. The myogenin promoter-luciferase construct was made by subcloning a 1.14 kB fragment of the myogenin promoter (containing the region from pGZ1092) from plasmid pGBB into pGL3-basic. The cardiac actin promoter-luciferase vector was generated by subcloning a fragment of the cardiac actin promoter from −440 to +6 into pGL3-basic.

Establishment and transient transfection of primary limb mesenchymal cultures

Limb mesenchymal cells were harvested from embryonic age 11.25-11.75 mouse embryos as previously described (19). To establish proximal or distal cultures the proximal half of each limb bud was separated from the distal half, and each pool of limb fragments was processed separately. The cells were resuspended at a density of 1.5−2×10⁷ cells/ml. For transfection purposes, cells were mixed with a DNA/FuGene6 mixture in a 2:1 ratio. FuGene6-DNA mixtures were prepared according to the manufacturer's instructions (Roche Biomolecular). Briefly, 1 μg of reporter, 1 μg of expression vector and 0.05 μg of pRLSV40 (Promega) were mixed for a total of ˜2 μg DNA in 100 μl of media and FuGene6. Fifty microlitres of the DNA mixture was transferred into a sterile 1.5 ml eppendorf tube, followed by 100 μl of cells. Cells were gently triturated and 10 μl was used to seed a single well of a 24-well culture dish. After 1.5 hrs in a humidified CO₂ incubator, 1 ml of media containing compounds of interest was added to each well and was subsequently replaced 24 hrs following transfection.

Analysis of reporter gene activity using the Dual Luciferase Assay System was done following the manufacturer's instructions (Promega). Briefly, ˜48 hrs post-transfection, cells were washed once with PBS and lysed in 100 μl of Passive Lysis Buffer for 20 min. Firefly and renilla luciferase activities were determined using 40 μl of lysate in a 96-well plate reading luminometer (Molecular Devices).

Cos P7 cells were maintained in DMEM supplemented with 10% FBS and transfected using the calcium phosphate method. EGFP-expressing cells were viewed using a Zeiss Axiovert 100S and images were captured with a DVC1300c color CCD camera.

Immunofluorescence and alcian blue staining of cultures

Alcian blue staining was performed on fixed cultures also as described (4). Mouse anti-MyHC monoclonal antibody supernatant was used to observe those cells within primary limb mesenchymal cultures that are myogenic (2). Briefly, cells were washed with PBS, fixed for 5 min. with cold methanol, rehydrated with PBS for 30 min. then incubated with the MF20 supernatant for 1 hour at room temperature (16). Following antibody incubation, cells were washed 3 times for 5 min. each prior to incubation in a goat anti-mouse IgG(H+L) Cy3-linked antibody (Jackson lmmunoresearch Laboratories, Pa.) at a 1:50 dilution in PBS for 30 min. Immunofluorescent cells were observed using a Zeiss Axiovert microscope, and images were captured by a DVC1300c colour digital camera using the Northern Exposure software program.

Northern blot analysis

Northern blots were carried out using total RNA from limb mesenchymal cultures, as previously described (19). Briefly, total RNA was extracted from cells 2 or 4 days after cultures were initiated. Cells were treated with media alone or with 10 ng/ml BMP-4. Synthesis of the Col II cDNA fragment used was as described previously (19). The Sox9 cDNA probe was made using an EST clone, GenBank accession number AI594348 (Research Genetics). The Sox9 fragment was released from pT7T3 using Eco RI and Not I.

### RESULTS AND DISCUSSION

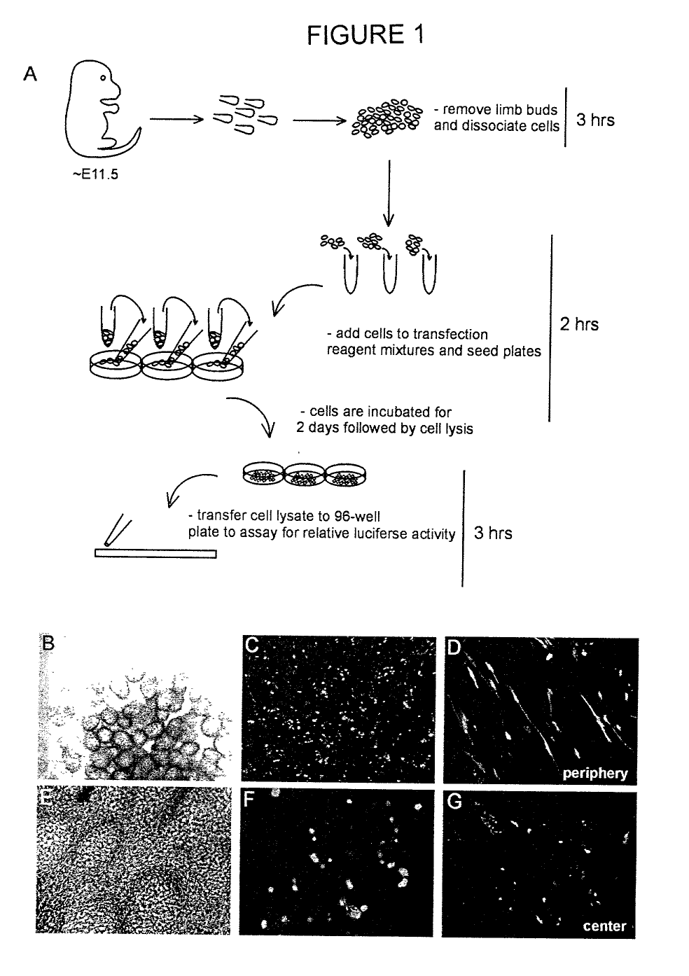

The methodology used to assess the ability of various factors (genes, compounds, drugs, etc.) to stimulate or repress chondrogenesis is illustrated in FIG. 1A. An in vitro assay that accurately reflects developmental processes requires the use of cells that closely follow the sequence of events occurring in vivo. In this respect, cultures derived from mouse embryonic limb mesenchyme have proven invaluable for the study of chondrogenesis. When plated at high density, limb mesenchymal cells of the appropriate embryonic stage form cartilage nodules in vitro in a manner that closely follows the in vivo process (1). Moreover, these cells are robust, easy to isolate, and are pluripotent. Shortly after seeding, the cells form a confluent monolayer with precartilaginous condensations evident within 24 hours. After approximately 48 hrs, small cartilage nodules appear that weakly stain with alcian blue, and cells weakly express Sox9 and col II. Following an additional 24-48 hrs there is a dramatic upregulation of Sox9 and col II expression which is closely associated with an increase in the number and size of cartilage nodules that stain intensely with alcian blue.

Given that cartilage differentiation is accompanied by the induction of col II by Sox9, an efficient assay that accurately monitors this induction would serve as an effective screen to identify factors that stimulate chondroblast differentiation. Several gene transfer approaches were initially considered, including the use of retroviral or adenoviral vectors, but only transient transfections allow for the potential analysis of hundreds to thousands of genes and/or compounds within a single experiment. In addition, when using transient transfections instead of retroviral or adenoviral vectors the time-consuming need to generate and package the virus is eliminated, again making the assay amenable to high-throughput screening. In addition, the inventors have developed optimal conditions under which we obtain a sufficient number of transfected cells within a relatively short period of time, allowing us to monitor cell fate changes that occur very early in the culture period.

To optimize transfection efficiency, a vector containing enhanced green fluorescent protein (EGFP) under the control of a constitutively active promoter was used. Initially cells were transfected after plating and media addition, however the distribution of transfected cells under these circumstances was found to be non-random, with many of the transfected cells appearing in the periphery of the culture—a region containing cells that do not efficiently undergo chondrogenesis. Subsequently, a method was developed that involves mixing the DNA/transfection reagent directly with the cell suspension prior to plating. The major advantages to the later approach are (a) the amount of DNA and transfection reagent required is kept to a minimum, and (b) there is a random distribution of EGFP-expressing cells within the culture. EGFP can be identified within many chondroblasts throughout the nodules, as well as within the periphery (FIGS. 1B-E). The expression of transiently transfected DNA is retained for a minimum of three days, providing ample time for the onset of cell differentiation.

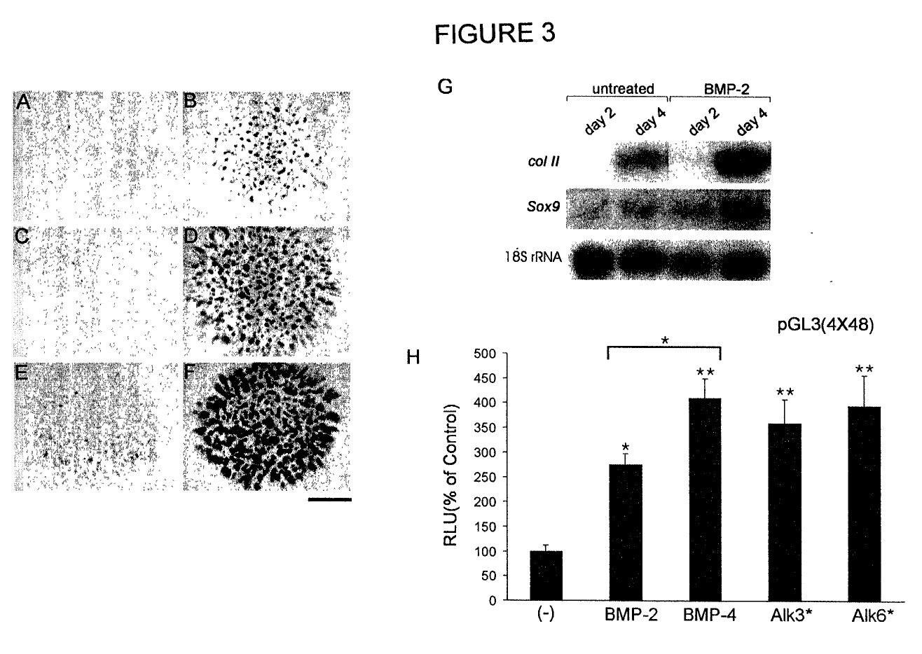

To follow Sox9 activity in the primary cultures, cells were transiently transfected with a pGL3(4X48) reporter gene. This construct differs from the previously described 4X48-p89 Col2a1 luciferase construct (14), in that the 4X48 enhancer element coupled to the col II minimal promoter was subcloned into pGL3-basic to increase the sensitivity of the reporter gene. Use of the reiterated 48bp enhancer element offers a much more sensitive system for following chondroblast differentiation compared to a single copy of the col II promoter (pW1-coIII-Luc) (FIG. 2A). Although the 4X48-p89 plasmid is highly sensitive to factors enhancing chondrogenesis, it responds weakly to basal levels of Sox9 within chondroprogenitors, making it an inefficient reporter for studying factors having a negative effect on Sox9 transactivation. The pGL3(4X48) reporter also responds dramatically to an increase in Sox9 (FIG. 2A), but has the added benefit of having a much higher output in untreated or unmodified cells (2B) enabling the study of genes or factors that modulate Sox9 negatively. The use of pGL3(4X48) reporter activity to follow cartilage differentiation was initially evaluated using bone morphogenetic proteins (BMPs) and components of the BMP signaling pathway. BMP-2 and-4 have been shown to potently stimulate chondrogenesis both in vitro and in vivo (5-8). In response to BMP-2 or -4, (10 ng/ml), nodules appear much earlier than normal and there is a dramatic increase in nodule number (FIGS. 3A-F). The ability of BMPs to enhance chondrogenesis is further demonstrated by an increase in Sox9 and col II expression in cultures treated with BMP-2 (FIG. 3G). Treatment of reporter gene-transfected cells with BMP-2 or BMP-4 leads to approximate 2-fold and 4.5-fold increases in reporter gene activity, respectively (FIG. 3H). These changes in reporter activity correlate with the abilities of BMP-2 and -4 to enhance the number and size of alcian blue-stained cartilage nodules compared to untreated cultures, in that BMP-4 is a much more potent inducer of chondrogenesis compared to BMP-2.

The utility of this reporter assay is largely dependent upon the ability to assess the influence of various genes on chondrogenesis at the single cell level. The influence of specific genes on endogenous Sox9 activity can be assessed by co-transfecting each gene of interest with the Sox9 reporter. In addition, the use of expression vectors containing wild-type, dominant-negative or constitutively active cDNAs either alone, or in combination, provides an efficient tool to precisely define the networks regulating chondrogenesis. To validate this approach, cells were co-transfected with constitutively active versions of the BMP type I receptors. The BMPs function through the binding and activation of BMPRI/II receptor complexes, and BMPs-2 and -4 have been shown to lead primarily to activation of either BMPRIA or 1B (17). In a previous study, BMPRIB was found to have a more important role in the early stages of chondrogenesis, particularly during condensation and chondroblast differentiation, compared to BMPRIA (23). Interestingly, using the assay described herein, expression of either BMPRIA or BMPRIB led to similar increases in reporter gene activity (FIG. 3G). Reporter gene activity also correlates with the induction of alcian blue stained nodules, demonstrating the usefulness of this method in predicting the effect of various factors on chondroblast differentiation.

In addition to members of the BMP signaling pathway, the inventors analyzed the ability of several other genes to modulate activity of the pGL3(4X48) luciferase reporter. Some of these genes have previously been found to influence the chondrogenic program (9, 10, 18, 21). Co-transfection of cells with various cDNA expression constructs has provided very useful information regarding the influence of specific factors on chondrogenesis (Table 1). Modulation of the pGL3(4X48) reporter gene is observed with several of the constructs tested. Further analysis of a subset of these constructs reveals that changes in reporter activity correlate with changes in alcian blue staining (data not shown). Of the many genes found to enhance reporter activity, Hoxd10 is of particular interest given that to date, no functional assay exists to examine Hox gene function during development, adding a further use for this assay. Table 1 highlights the use of this assay as a powerful means through which regulators of chondroblast differentiation can be identified, and their role within the network of factors regulating chondrogenesis defined.

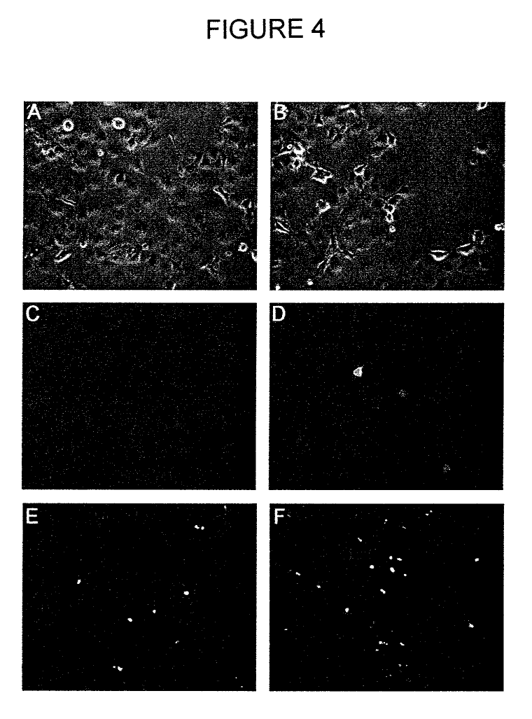

To examine Sox9 activation in single cells an EGFP-based reporter was constructed from pGL3(4X48), to generate pGL3(4X48)-EGFP. To confirm the absolute requirement of Sox9 in the activation of this reporter construct, Cos P7 cells, which have no chondrogenic capacity, were transfected with a pGL3(4X48)-EGFP reporter construct. Given the origin of Cos P7 cells it is unlikely that they express significant levels of Sox9. It is not surprising therefore, that no fluorescing cells can be seen following transfection of these cells with the EGFP reporter construct unless they were co-transfected with a Sox9 expression vector (FIGS. 4A-D). In contrast, EGFP expression is seen in primary limb bud cultures, which differentiate into cartilage spontaneously and the number of fluorescing cells is enhanced in response to the chondrogenic stimulator, BMP-4 (FIGS. 4E, F). Given that the EGFP reporter is not activated in the absence of Sox9, it is reasonable to assume that anything that increases EGFP expression does so indirectly, through increased expression or activation of Sox9. Thus, the use of this promoter construct provides an authentic and accurate measure of Sox9 activity at the single cell level, and ultimately of chondroblast differentiation.

The simplicity of this assay enables the rapid identification of genes and/or factors that regulate the chondrogenic program. The inventors have used this methodology to identify several genes that both inhibit and augment chondrogenesis and are now applying this approach to defining mechanisms through which chondrogenic stimulatory factors exert their effects. Using this strategy it is possible to not only identify genes involved in this differentiation program, but also to organize them into a network. The ability to efficiently transfect primary cultures and follow the activity of transfected reporters will be an extremely useful tool, as researchers attempt to make sense of the overwhelming amount of sequence information that is becoming available. Various factors including genes, compounds, and potential drugs, can be screened for their importance in not only the chondrogenic process, but other developmental programs as well. For instance, we have used muscle-specific reporters to monitor changes in myogenesis within limb mesenchymal cultures. In this manner, switches controlling decisions between more than one possible cell fate can be identified. The identification of such switches will enhance our understanding of early developmental processes and advance the field of tissue engineering, which relies extensively on identifying suitable conditions under which developmental or reparative processes can be recapitulated in vitro.

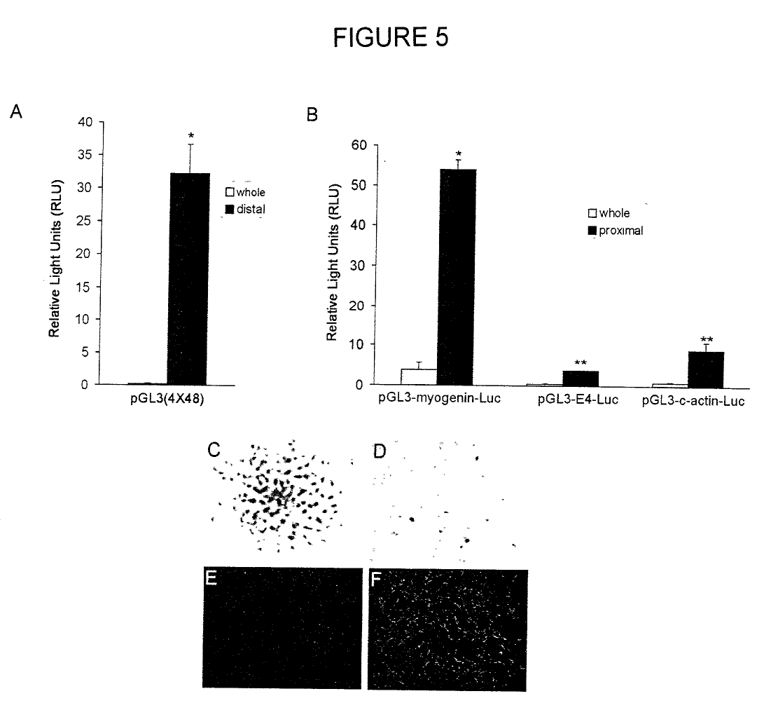

The heterogeneous nature of the primary limb mesenchymal cultures provides a relevant system for studying cell differentiation in the context of normal embryonic development, during which no developmental program occurs in isolation. However, in some instances, the assay has been modified such that the population of cells used is enriched for specific precursors. To enhance the portion of muscle precursors available, the proximal portion of the limb bud, which contains considerably more somitic-derived myogenic cells, is preferentially dissected. In contrast, the distal portion can be used as a source that is enhanced for cartilage precursors. Basal activity of the pGL3(4X48) reporter is substantially greater in cells of the distal portion of the developing limb compared to those of the whole limb (FIG. 5A). Accordingly, muscle-specific reporters containing the myogenin promoter (pGL3-myogenin-Luc), the cardiac actin promoter (pGL3-c-actin-Luc), or 4 repeats of an E box, the consensus sequence bound by all the myogenic regulatory factors, (pGL2-E4-Luc) are all substantially more active in cells from the proximal portion of the limb bud. This method of preferentially using specific regions of the limb serves two major purposes (a) variability in measurements is reduced due to a less heterogeneous cell population and (b) having such high basal reporter activity provides another means by which to observe negative influences on reporter activity. The differences in precursor populations of proximal versus distal limb mesenchyme is also reflected in culture, as there are many more myocytes within proximal cultures, detected by immunofluorescence with an anti-myosin heavy chain (MyHc) antibody, and more cartilage formation in distal cultures (FIGS. 5B-E). Although this slight modification is useful for overcoming some of the obstacles associated with this assay, the whole limb preparations are still beneficial, particularly for studying the interplay between multiple differentiation programs. As such, the inventors are currently using this assay with and without the modifications described to better understand the importance of factors in both muscle and cartilage formation, as these two events take place in close proximity to one another and utilize many common factors.

Here the inventors have described an assay that offers an effective measure of cell differentiation. As a proof-of-principle, the inventors have shown that this technology accurately reflects what is known to date about the role of the BMP signaling pathway in chondrogenesis. The inventors have tested several other factors of interest for their ability to modulate col II expression and in doing so have begun to delineate the functions of these factors during cartilage formation. Through the use of various inhibitors and activators of signaling pathways, this technology will allow for the elucidation of the mechanisms through which these functions are achieved. Thus, this high-content screen is a timely addition to the tools that can be used to fill in many of the gaps that exist in our understanding of critical cellular processes. Furthermore, the screen can be used to identify modulators of chondrogenesis that have therapeutic potential.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Modulation of pGL3(4X48) reporter gene activity by various factors

| | normalized | | normalized |
|---|---|---|---|
| Gene/protein | RLU¹ | Gene/Protein | RLU |
| Wnt pathway | | AP-1 family | |
| Wnt3a | 0.5 | c-Fos | 0.6 |
| Wnt7a | 0.4 | FosB | 0.8 |
| β-catenin² | 0.2 | Fra2 | 1.6 |
| Axin-2 | 1.0 | dnFos | 0.8 |
| Frzb1 | 1.1 | c-Jun | 0.7 |
| | | JunB | 1.4 |
| BMP pathway | | JunD | 1.3 |
| BMP-2 (10 ng/ml) | 1.9 | Kinases³ | |
| BMP-4 (10 ng/ml) | 4.2 | | |
| Noggin (100 ng/ml) | 0.3 | cMEK1 | 0.6 |
| cBMPRIA | 3.8 | cMEKK1 | 0.5 |
| cBMPRIB | 4.2 | cMKK6 | 3.1 |
| | | PKAc⁴ | 1.8 |
| Other | | dnSEK1 | 0.6 |
| | | dnJNK1 | 0.7 |
| Hoxd10 | 4.0 | MKK7 | 0.9 |
| NCoR | 1.4 | MLK3 | 0.9 |
| shh | 4.8 | cMEKK1 + dnFos | 7.3 |
| BS69 | 2.1 | | |
| RARγ1 | 0.5 | | |

¹RLU values were normalized to control reporter activity.
²a stablized version of β-catenin.
³c preceding gene name indicates a constitutively active version of the kinase.
⁴catalytic subunit of protein kinase A.

### FULL CITATIONS FOR REFERENCES REFERRED TO IN THE SPECIFICATION

1. Ahrens, P. B., M. Solursh, and R. S. Reiter 1977. Stage related capacity for limb chondrogenesis in cell culture Dev. Biol. 60:69-82.

2. Bader, D., T. Masaki, and D. A. Fischman 1982. Immunochemical analysis of myosin heavy chain during avian myogenesis in vivo and in vitro J Cell Biol. 95:763-70.

3. Bi, W., J. M. Deng, Z. Zhang, R. R. Behringer, and B. de Crombrugghe 1999. Sox9 is required for cartilage formation Nature Genetics. 22:85-9.

4. Cash, D. E., C. Bock, K. Schughart, E. Linney, and T. M. Underhill 1997. Retinoic acid receptor a function in vertebrate limb skeletogenesis: a modulator of chondrogenesis J. Cell Biol. 136:445-457.

5. Chen, P., J. L. Carrington, R. G. Hammonds, and A. H. Reddi 1991. Stimulation of chondrogenesis in limb bud mesoderm cells by recombinant human bone morphogenetic protein 2B (BMP-2B) and modulation by transforming growth factor β₁ and β₂ Exp. Cell Res. 195:509-515.

6. Denker, A. E., A. R. Haas, S. B. Nicoll, and R. S. Tuan 1999. Chondrogenic differentiation of murine C3H1OT1/2 multipotential mesenchymal cells: I. Stimulation by bone morphogenetic protein-2 in high-density micromass cultures Differentiation. 64:67-76.

7. Duprez, D., E. J. de H. Bell, M. K. Richardson, C. W. Archer, L. Wolpert, P. M. Brickell, and P. H. Francis-West 1996. Overexpression of BMP-2 and BMP-4 alters the size and shape of developing skeletal elements in the chick limb Mech Dev. 57:145-157.

8. Duprez, D. M., M. Coltey, H. Amthor, P. M. Brickell, and C. Tickle 1996. Bone Morphogenetic Protein-2 (BMP-2) inhibits muscle development and promotes cartilage formation in chick limb bud cultures Dev Biol. 174:448-452.

9. Enomoto-lwamoto, M., T. Nakamura, T. Aikawa, Y. Higuchi, T. Yuasa, A. Yamaguchi, T. Nohno, S. Noji, T. Matsuya, K. Kurisu, E. Koyama, M. Pacifici, and M. Iwamoto 2000. Hedgehog proteins stimulate chondrogenic cell differentiation and cartilage formation J Bone Miner Res. 15:1659-68.

10. Goff, D. J., and C. J. Tabin 1997. Analysis of Hoxd-13 and Hoxd-11 misexpression in chick limb buds reveals that Hox genes affect both bone condensation and growth. Development. 124:627-636.

11. Hall, B. K., and T. Miyake 1995. Divide, accumulate, differentiate: cell condensation in skeletal development revisited Int. J. Dev. Biol. 39:881-893.

12. Johnson, R. L., and C. J. Tabin 1997. Molecular models for vertebrate limb development Cell. 90:979-990.

13. Lefebvre, V., W. Huang, V. R. Harley, P. N. Goodfellow, and B. de Crombrugghe 1997. SOX9 is a potent activator of the chondrocyte-specific enhancer of the pro alpha1 (II) collagen gene Mol. Cell. Biol. 17:2336-2346.

14. Lefebvre, V., G. Zhou, K. Mukhopadhyay, C. N. Smith, Z. Zhang, H. Eberspaecher, X. Zhou, S. Sinha, S. N. Maity, and B. de Crombrugghe 1996. An 18-base-pair sequence in the mouse Pro-alpha-1(II) collagen gene is sufficient for expression in cartilage and binds nuclear proteins that are selectively expressed in chondrocytes Molecular and Cellular Biology. 16:4512-4523.

15. Murakami, S., M. Kan, W. L. McKeehan, and B. de Crombrugghe 2000. Up-regulation of the chondrogenic Sox9 gene by fibroblast growth factors is mediated by the mitogen-activated protein kinase pathway Proc Natl Acad Sci U S A. 97:1113-8.

16.Ridgeway, A. G., S. Wilton, and I. S. Skerjanc 2000. Myocyte enhancer factor 2C and myogenin up-regulate each other's expression and induce the development of skeletal muscle in P19 cells J Biol Chem. 275:41-6.

17. Rosen, V., R. S. Thies, and K. Lyons 1996. Signaling pathways in skeletal formation: A role for BMP receptors Ann NY Acad Sci. 785:59-69.

18. Rudnicki, J. A., and A. M. Brown 1997. Inhibition of chondrogenesis by Wnt gene expression in vivo and in vitro Dev Biol. 185:104-18.

19. Weston, A., V. Rosen, R. A. S. Chandraratna, and T. M. Underhill 2000. Regulation of skeletal progenitor differentiation by the BMP and retinoid signaling pathways. J. Cell Biol. 148:679-690.

20. Wright, E., M. R. Hargrave, J. Christiansen, L. Cooper, J. Kun, T. Evans, U. Gangadharan, A. Greenfield, and P. Koopman 1995. The Sry-related gene Sox9 is expressed during chondrogenesis in mouse embryos Nature Genet. 9:15-20.

21. Yokouchi, Y., S. Nakazato, M. Yamamoto, Y. Goto, T. Kameda, H. Iba, and A. Kuroiwa 1995. Misexpression of Hoxa-13 induces cartilage homeotic transformation and changes cell adhesiveness in chick limb buds Genes & Dev. 9:2509-2522.

22. Zhou, G., S. Garofalo, K. Mukhopadhyay, V. Lefebvre, C. N. Smith, H. Eberspaecher, and B. de Crombrugghe 1995. A 182 bp fragment of the mouse proα1(II) collagen gene is sufficient to direct chondrocyte expression in transgenic mice Journal of Cell Science. 108:3677-3684.

23. Zou, H., R. Wieser, J. Massague, and L. Niswander 1997. Distinct roles of type I bone morphogenetic protein receptors in the formation and differentiation of cartilage Genes & Dev. 11:2191-2203.

We claim:

1. A method of identifying a modulator of chondrogenesis comprising:
(a) providing cells capable of differentiating into chondroblasts or chondrocytes;
(b) transfecting the cells with a nucleic acid construct comprising a reporter gene that is detectable upon chondroblast or chondrocyte differentiation;
(c) adding a test compound to the transfected cells; and
(d) determining the effect of the test compound on chondroblast or chondrocyte differentiation.

2. A method according to claim 1 wherein the cells are mesenchymal cells.

3. A method according to claim 1 wherein the cells are limb mesenchymal cells.

4. A method according to claim 1 wherein the reporter gene encodes an enhancer element that binds to a transcription factor whose activity changes during chondroblast differentiation.

5. A method according to claim 4 wherein the reporter gene is responsive to the transcription factor Sox9.

6. A method according to claim 5 wherein the nucleic acid construct comprises at least one Sox9 binding site, a promoter and a detectable marker.

7. A method according to claim 6 wherein the nucleic acid construct contains four Sox9 binding sites.

8. A method according to claim 6 wherein the marker encodes a luciferase or an enhanced green fluorescent protein.

9. A method according to claim 6 wherein the nucleic acid construct is pGL3(4X48)-luciferase as shown in FIG. 6.

10. A method according to claim 6 wherein the nucleic acid construct is pGL3(4X48)-enhanced green fluorescent protein as shown in FIG. 6.

11. A method according to claim 1 wherein the transfection is a transient transfection.

12. A method according to claim 1 wherein the cells are plated at high density.

13. A method according to claim 1 wherein the test compound is a nucleic acid molecule comprising a test gene that is transfected into the cells.

14. A nucleic acid construct which is pGL3(4X48)-luciferase as shown in FIG. 6.

15. A nucleic acid construct which is pGL3(4X48)-enhanced green fluorescent protein as shown in FIG. 6.

16. A kit for identifying modulators of chondrogenesis comprising a nucleic acid construct comprising a reporter gene that is responsive to the transcription factor Sox9 and instructions for the use thereof.

17. A kit according to claim 16 comprising the nucleic acid construct pGL3 (4X48)-luciferase as shown in FIG. 6.

18. A kit according to claim 16 comprising the nucleic acid construct pGL3 (4X48)-enhanced green fluorescent protein as shown in FIG. 6.

19. A method of diagnosing or monitoring a disease that is associated with a modulation in chondrogenesis comprising:
(a) providing cells capable of differentiating into chondroblasts or chondrocytes;
(b) transfecting the cells with a nucleic acid construct comprising a reporter gene that is detectable upon chondroblast or chondrocyte differentiation;
(c) adding a test sample to the transfected cells; and
(d) determining the effect of the test sample on chondroblast or chondrocyte differentiation.

20. A method according to claim 19 wherein the test sample is a bodily fluid.

21. A method according to claim 19 wherein the bodily fluid is synovial fluid.

22. A method of conducting a drug discover y business comprising:
(a) providing one or more assay systems for identifying a modulator of chondrogenesis;
(b) conducting therapeutic profiling of modulators identified in step (a), or further analogs thereof, for efficacy and toxicity in animals; and
(c) formulating a pharmaceutical preparation including one or more modulators identified in step (b) as having an acceptable therapeutic profile.

23. A method of conducting a target discovery business comprising:
(a) providing one or more assay systems for identifying modulators of chondrogenesis;
(b) (optionally) conducting therapeutic profiling of modulators identified in step (a) for efficacy and toxicity in animals; and
(c) licensing, to a third party, the rights for further drug development and/or sales for modulators identified in step (a), or analogs thereof.

24. A modulator of chondrogenesis identified according to the method of claim 1.

25. A pharmaceutical composition for use in the modulation of chondrogenesis comprising a modulator of chondrogenesis identified according to the method of claim 1.

26. A method of making a pharmaceutical composition for use in modulating chondrogenesis comprising mixing a modulator of chondrogenesis identified according to a method of claim 1 with a suitable diluent or carrier.
